(19) United States
(12) Patent Application Publication
(10) Pub. No.: US 2024/0108393 A1
(43) Pub. Date: Apr. 4, 2024

(54) THERMAL CUTTING ELEMENTS FOR ELECTROSURGICAL INSTRUMENTS AND ELECTROSURGICAL INSTRUMENTS AND SYSTEMS INCORPORATING THE SAME

(71) Applicant: Covidien LP, Mansfield, MA (US)

(72) Inventors: James D. Allen, IV, Broomfield, CO (US); William E. Robinson, Boulder, CO (US)

(21) Appl. No.: 18/274,997

(22) PCT Filed: Jan. 11, 2022
(86) PCT No.: PCT/US2022/011910

§ 371 Date: Jul. 29, 2023

Related U.S. Application Data
(60) Provisional application No. 63/144,002, filed on Feb. 1, 2021.

(51) Int. Cl. A61B 18/08; A61B 18/10

(52) U.S. Cl. CPC A61B 18/085; A61B 18/10; A61B 2018/00601

(57) ABSTRACT

A thermal cutting element of an electrosurgical instrument includes a base substrate defining a first side and a second side, at least one insulating layer disposed on at least the first side of the base substrate, and a heater circuit trace disposed on the at least one insulating layer. The thermal cutting element has an operating temperature of at least about 350° C. Temperature Coefficient of Resistance (TCR) of at least about 50 ppm/° C. An electrosurgical instrument and electrosurgical system including the thermal cutting element are also provided.

### CROSS-REFERENCE TO RELATED APPLICATIONS

This application is a 371 National Stage Application of International Application No. PCT/US2022/011910, filed Jan. 11, 2022, which claims the benefit of, and priority to, U.S. Provisional Patent Application No. 63/144,002 filed on Feb. 1, 2021, the entire contents of which are hereby incorporated herein by reference.

### FIELD

The present disclosure relates to surgical instruments and systems and, more particularly, to thermal cutting elements for electrosurgical instruments and electrosurgical instruments and systems incorporating the same.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy-based tissue cutting element may be provided to cut the treated tissue using energy, e.g., thermal, electrosurgical, ultrasonic, light, or other suitable energy.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

Provided in accordance with aspects of the present disclosure is a thermal cutting element of an electrosurgical instrument including a base substrate defining a first side and a second side, at least one insulating layer disposed on at least the first side of the base substrate, and a heater circuit trace disposed on the at least one insulating layer. The thermal cutting element has an operating temperature of at least about 350° C. and a Temperature Coefficient of Resistance (TCR) of at least about 50 ppm/° C.

In an aspect of the present disclosure, the thermal cutting element has a TCR of at least about 900 ppm/° C. In another aspect, the TCR is at least about 3000 ppm/° C.

In an aspect of the present disclosure, the operating temperature is at least about 400° C. In another aspect, the operating temperature is at least about 450° C.

In an aspect of the present disclosure, a difference between a resistance of the thermal cutting element at room temperature and at the operating temperature is from about 10 ohms to about 1000 ohms. In another aspect, the difference is from about 20 ohms to about 400 ohms.

In an aspect of the present disclosure, an applied voltage of from about 10 volts to about 175 volts is required to at least one of heat the thermal cutting element to the operating temperature or maintain the thermal cutting element at the operating temperature. In another aspect, the required applied voltage is from about 25 volts to about 100 volts.

In an aspect of the present disclosure, the thermal cutting element further includes an encapsulating layer disposed on the first side of the base substrate.

An electrosurgical instrument provided in accordance with aspects of the present disclosure includes first and second jaw members each defining a tissue treating surface. The first and second jaw members are pivotably coupled to one another such that at least one of the first or second jaw members is movable relative to the other from a spaced-apart position to an approximated position to grasp tissue between the tissue treating surfaces. The tissue treating surfaces are adapted to connect to a source of energy to treat tissue grasped therebetween. One of the first or second jaw members includes a thermal cutting element extending from the tissue treating surface thereof. The thermal cutting element may be configured similar to any of the aspects detailed above or otherwise herein.

An electrosurgical system provided in accordance with the present disclosure includes an electrosurgical generator and a thermal cutting element configured similar to any of the aspects detailed above or otherwise herein. The electrosurgical generator is configured to supply an AC voltage signal to the heater circuit trace of the thermal cutting element to at least one of heat the thermal cutting element to the operating temperature or maintain the thermal cutting element at the operating temperature.

In an aspect of the present disclosure, the electrosurgical generator is configured to monitor power during the supply of the AC voltage signal and to at least one of provide an indicator or stop the AC voltage signal when the power declines to a threshold power level.

In another aspect of the present disclosure, the electrosurgical generator is configured to monitor power slope during the supply of the AC voltage signal and to at least one of provide an indicator or stop the AC voltage signal when the power slope flattens to a power slope threshold.

In still another aspect of the present disclosure, the AC voltage signal has an applied voltage of from about 10 volts to about 175 volts.

In yet another aspect of the present disclosure the electrosurgical generator is configured to provide a maximum power associated with the AC voltage signal of about 50 W.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure shown connected to an electrosurgical generator;

FIG. 2 is a perspective view of a hemostat-style electrosurgical forceps provided in accordance with the present disclosure;

FIG. 3 is a schematic illustration of a robotic surgical instrument provided in accordance with the present disclosure;

FIG. 4 is a perspective view of an end effector assembly of the forceps of FIG. 1 including first and second jaw members;

FIG. 5 is a perspective view of the thermal cutting element of the second jaw member of the end effector assembly of FIG. 4; and

FIG. 6 is an exemplary power versus time graph illustrating cut completion determination in accordance with the present disclosure.

### DETAILED DESCRIPTION

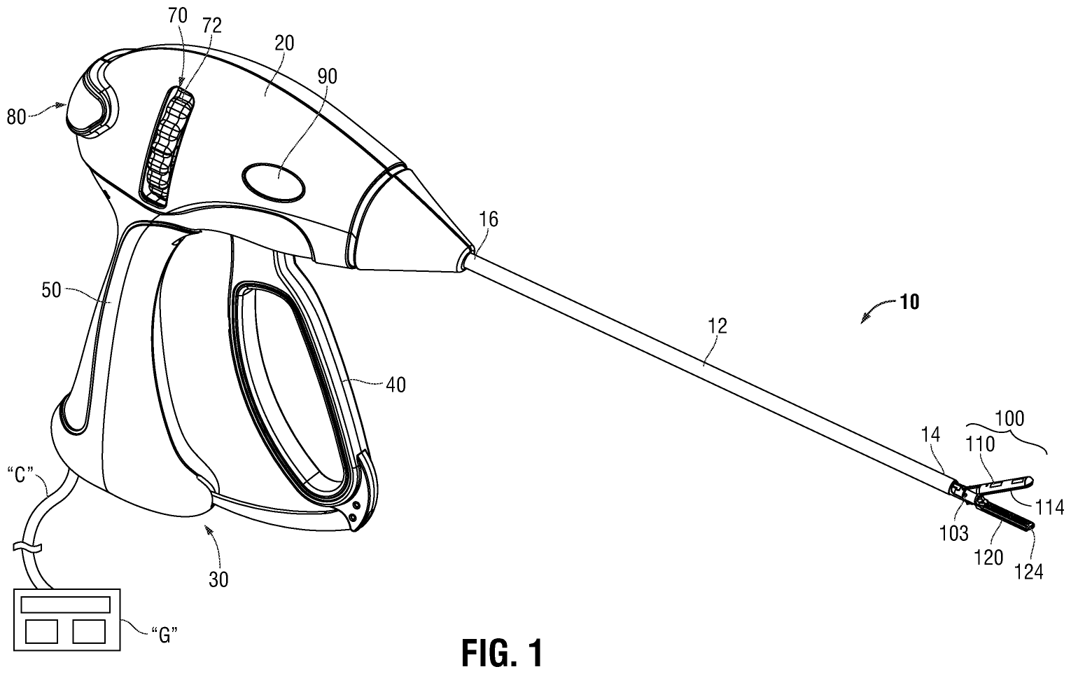

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

Forceps 10 includes a housing 20, a handle assembly 30, a rotating assembly 70, a first activation switch 80, a second activation switch 90, and an end effector assembly 100. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable “C” that connects forceps 10 to an energy source, e.g., an electrosurgical generator “G.” Cable “C” includes a wire (or wires) (not shown) extending therethrough that has sufficient length to extend through shaft 12 in order to connect to one or both tissue treating surfaces 114, 124 of jaw members 110, 120, respectively, of end effector assembly 100 to provide energy thereto. Alternatively, forceps 10 may be configured as a cordless device such as, for example, including an on-board power source, e.g., a DC battery, and an on-board electrosurgical generator powered by the on-board power source. The power source may provide energy to thermal cutting element 130 while the electrosurgical generator provides electrosurgical energy to tissue treating surfaces 114, 124, although the electrosurgical generator may also provide the energy to energy to thermal cutting element 130. In other configurations, forceps 10 includes an on-board power source for providing energy to thermal cutting element 130 and connects to electrosurgical generator “G” via cable “C” for providing electrosurgical energy to tissue treating surfaces 114, 124.

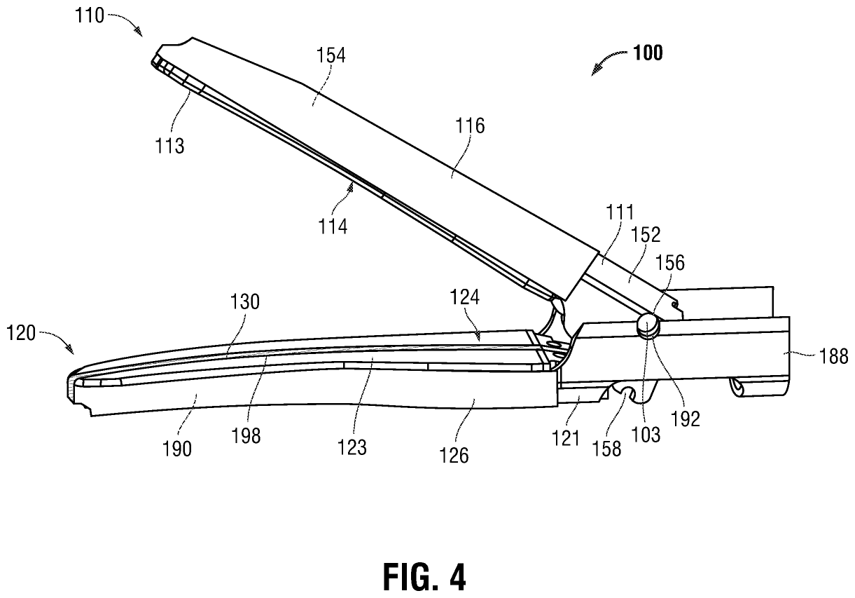

First activation switch 80 is coupled to tissue treating surfaces 114, 124 and the electrosurgical generator “G” for enabling the selective activation of the supply of energy to jaw members 110, 120 for treating, e.g., cauterizing, coagulating/desiccating, and/or sealing, tissue. Second activation switch 90 is coupled to thermal cutting element 130 of jaw member 120 (FIG. 4) and the electrosurgical generator “G” for enabling the selective activation of the supply of energy to thermal cutting element 130 for thermally cutting tissue.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced apart position and an approximated position to grasp tissue between tissue treating surfaces 114, 124 of jaw members 110, 120. As shown in FIG. 1, movable handle 40 is initially spaced apart from fixed handle 50 and, correspondingly, jaw members 110, 120 of end effector assembly 100 are disposed in the spaced apart position. Movable handle 40 is depressible from this initial position towards fixed handle 50 to a depressed position corresponding to the approximated position of jaw members 110, 120. Rotating assembly 70 includes a rotation wheel 72 that is selectively rotatable in either direction to correspondingly rotate shaft 12 and end effector assembly 100 relative to housing 20.

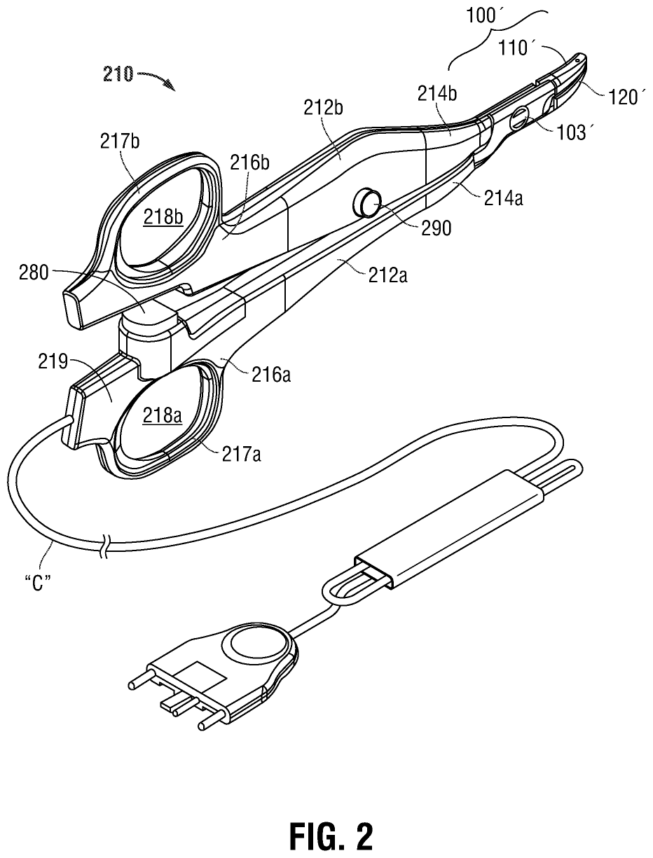

Referring to FIG. 2, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to and including any of the features of end effector assembly 100 (FIGS. 1 and 4). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212a, includes a proximal shaft connector 219 configured to connect forceps 210 to a source of energy, e.g., electrosurgical generator “G” (FIG. 1). Proximal shaft connector 219 secures a cable “C” to forceps 210 such that the user may selectively supply energy to jaw members 110′, 120′ for treating tissue. More specifically, a first activation switch 280 is provided on one of the shaft members, e.g., shaft member 212a, for supplying energy to jaw members 110′, 120′ to treat tissue upon sufficient approximation of shaft members 212a, 212b, e.g., upon activation of first activation switch 280 via the other shaft member 212b. A second activation switch 290 disposed on either or both of shaft members 212a, 212b is coupled to the thermal cutting element (not shown, similar to thermal cutting element 130 of jaw member 120 (FIG. 4)) of one of the jaw members 110′, 120′ of end effector assembly 100′ and to the electrosurgical generator “G” for enabling the selective activation of the supply of energy to the thermal cutting element for thermally cutting tissue.

Jaw members 110′, 120′ define a curved configuration wherein each jaw member is similarly curved laterally off of a longitudinal axis of end effector assembly 100′. However, other suitable curved configurations including curvature towards one of the jaw members 110′, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

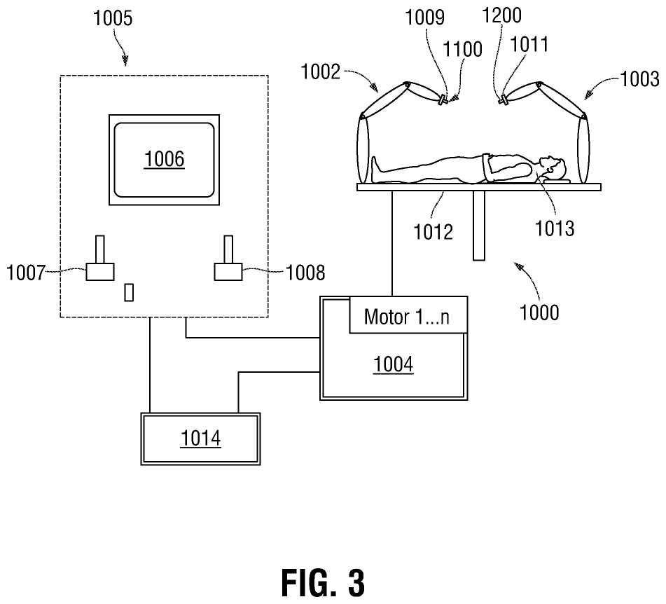

Referring to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in an operating mode. Robotic surgical instrument 1000 may be configured for use on a patient 1013 lying on a patient table 1012 to be treated in a minimally invasive manner. Robotic surgical instrument 1000 may further include or be capable of accessing a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to and may include any of the features of end effector assembly 100 (FIGS. 1 and 4), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

Turning to FIG. 4, end effector assembly 100, as noted above, includes first and second jaw members 110, 120. Either or both jaw members 110, 120 may include a structural frame 111, 121, an insulative spacer (not shown), a tissue treating plate 113, 123 defining the respective tissue treating surface 114, 124 thereof, and, in aspects, an outer insulative jacket 116, 126. Tissue treating plates 113, 123 may be pre-formed and engaged with the insulative spacers and/or other portion(s) of jaw members 110, 120 via, for example, overmolding, adhesion, mechanical engagement, etc., or may be deposited onto the insulative spacers, e.g., via sputtering or other suitable deposition technique.

Jaw member 110, as noted above, includes a structural frame 111, an insulative spacer (not shown), a tissue treating plate 113 defining tissue treating surface 114, and, in aspects, an outer insulative jacket 116. Structural frame 111 may be formed from stainless steel or other suitable material configured to provide structural support to jaw member 110. Structural frame 111 includes a proximal flange portion 152 about which jaw member 110 is pivotably coupled to jaw member 120 via pivot 103 and a distal body portion 154 that supports the other components of jaw member 110, e.g., the insulative spacer, tissue treating plate 113, and outer insulative jacket 116 (where provided). In shaft-based or robotic configurations, proximal flange portion 152 enables operable coupling of jaw member 110 to the drive assembly (not shown) to enable pivoting of jaw member 110 relative to jaw member 120 in response to actuation of the drive assembly. More specifically, proximal flange portion 152 may define an aperture 156 for receipt of pivot 103 and at least one catch 158 for receipt of a drive pin of the drive assembly (not shown) such that translation of the drive pin, e.g., in response to actuation of movable handle 40 (FIG. 1) or a robotic drive, pivots jaw member 110 about pivot 103 and relative to jaw member 120 between the spaced apart position and the approximated position. However, other suitable drive arrangements are also contemplated, e.g., using cam pins and cam slots, a screw-drive mechanism, etc. In hemostat-style devices, proximal flange portion 152 is secured to one of the shaft members, e.g., shaft member 212a of forceps 210 (see FIG. 2). Proximal flange portion 152 may be bifurcated to define a pair of spaced apart proximal flange portion segments or may otherwise be configured.

Distal body portion 154 of structural frame 111 extends distally from proximal flange portion 152 to support the other components of jaw member 110. The insulative spacer of jaw member 110 is supported on distal body portion 154 of structural frame 111 and is formed from an electrically insulative material capable of withstanding high temperatures such as, for example, up to at least 400° C., although other configurations are also contemplated. The insulative spacer may be formed from ceramic or other suitable material, e.g., PTFE, PEEK, PEI, etc. Tissue treating plate 113 is supported or received on the insulative spacer and is electrically connected, e.g., via one or more electrical leads (not shown), to first activation switch 80 (FIG. 1) and electrosurgical generator “G” (FIG. 1) to enable selective energization of tissue treating plate 113, e.g., as one pole of a bipolar Radio Frequency (RF) electrosurgical circuit. However, other suitable energy modalities, e.g., thermal, ultrasonic, light, microwave, infrared, etc., are also contemplated. The insulative spacer serves to electrically isolate structural frame 111 and tissue treating plate 113 from one another.

Continuing with reference to FIG. 4, jaw member 120 includes a structural frame 121, an insulative spacer (not shown), a tissue treating plate 123 defining tissue treating surface 124, and, in aspects, an outer insulative jacket 126. Jaw member 120 further include thermal cutting element 130. Structural frame 121 of jaw member 120 defines a proximal flange portion 188 and a distal body portion 190 extending distally from proximal flange portion 188. Proximal flange portion 188 may be bifurcated to define a pair of spaced apart proximal flange portion segments or may define any other suitable configuration. Proximal flange portion 188 of jaw member 120 and proximal flange portion 152 of jaw member 110 may define a nestled configuration, e.g., wherein one of the proximal flange portions 152, 188 is received within the other, an overlapping configuration, e.g., wherein proximal flange portions 152, 188 at least partially overlap one another, or an offset configuration, e.g., wherein proximal flange portions 152, 188 are positioned in side-by-side relation. Regardless of the particular arrangement of proximal flange portions 152, 188, proximal flange portion 188 further defines a cut out 192 configured for receipt of pivot 103, e.g., welded or otherwise secured therein, to pivotably couple jaw members 110, 120 with one another. Proximal flange portion 188 may be secured to shaft 12 (FIG. 1) in shaft-based configurations (or a corresponding shaft portion in robotic configurations); alternatively, a bilateral configuration may be provided whereby both jaw member 110 and jaw member 120 are pivotable relative to shaft 12 (FIG. 1). In hemostat-style configurations, proximal flange portion 188 may be secured to elongated shaft 212b (FIG. 2).

The insulative spacer of jaw member 120 is supported on distal body portion 190 of structural frame 121 and is formed from an electrically insulative material capable of withstanding high temperatures such as, for example, up to at least 400° C., although other configurations are also contemplated. The insulative spacer may be formed from ceramic or other suitable material, e.g., PTFE, PEEK, PEI. Tissue treating plate 123 is supported or received on the insulative spacer. Tissue treating plate 123, in particular, defines a longitudinally extending slot 198 therethrough along at least a portion of the length thereof. Slot 198 may be transversely centered on tissue treating surface 124 or may be offset relative thereto and may be linear, curved, include angled sections, etc. similarly or differently from the configuration, e.g., curvature, of jaw member 120. Slot 198 exposes a portion of thermal cutting element 130, which may be recessed relative to tissue treating surface 124, substantially co-planar with tissue treating surface 124, or protrude beyond tissue treating surface 124 towards jaw member 110. In aspects where thermal cutting element 130 protrudes, thermal cutting element 130 may contact an opposing portion of jaw member 110 to set a minimum gap distance, e.g., of from about 0.001 inches to about 0.006 inches, between tissue treating surfaces 114, 124 in the approximated position of jaw members 110, 120.

Tissue treating plate 123 is electrically connected, e.g., via one or more electrical leads (not shown), to first activation switch 80 (FIG. 1) and electrosurgical generator “G” (FIG. 1) to enable selective energization of tissue treating plate 123, e.g., as the other pole of the bipolar (RF) electrosurgical circuit including tissue treating plate 113. In this manner, in the approximated position of jaw members 110, 120 grasping tissue therebetween, bipolar RF electrosurgical energy may be conducted between tissue treating plates 113, 123 and through the grasped tissue to treat, e.g., seal, the grasped tissue. However, other suitable energy modalities, e.g., thermal, ultrasonic, light, microwave, infrared, etc., are also contemplated, as are other suitable tissue treatments, e.g., coagulation.

Thermal cutting element 130 may be secured within and directly to the insulative spacer 122 of jaw member 120 in any suitable manner, e.g., adhesive, friction fitting, overmolding, mechanical engagement, etc., or may be indirectly secured relative to the insulative spacer (in contact with or spaced apart therefrom) via attachment to one or more other components of jaw member 120. Alternatively, the insulative spacer may be omitted and thermal cutting element 130 secured within jaw member 120 (to one or more components thereof) in any other suitable manner. Other suitable configurations for supporting thermal cutting element 130 within jaw member 120 are also contemplated. Thermal cutting element 130 may protrude distally beyond the distal tip of the insulative spacer of jaw member 120 (thus defining the distal-most extent of jaw member 120), may be substantially flush therewith, or may be recessed relative thereto. In aspects where end effector assembly 100, or a portion thereof, is curved, thermal cutting element 130 may similarly be curved.

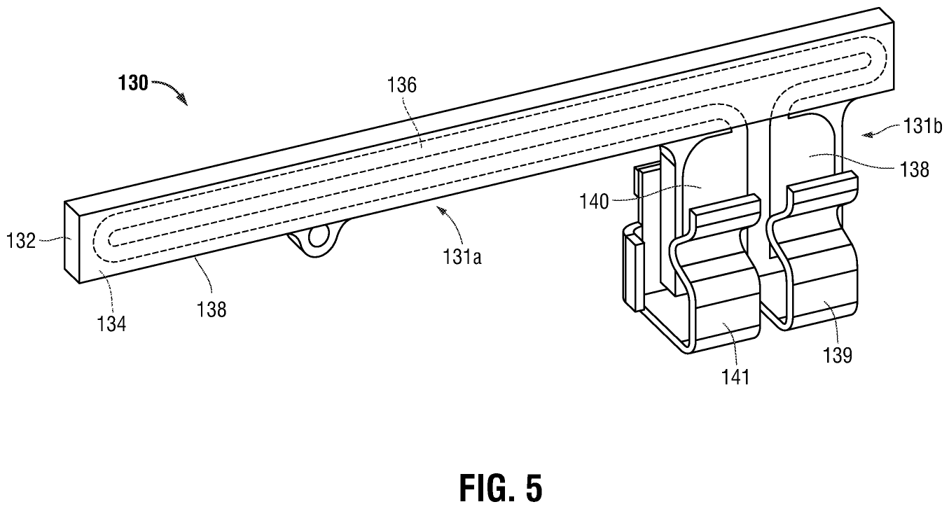

With additional reference to FIG. 5, thermal cutting element 130 includes a body 131a and a proximal extension 131b. Thermal cutting element 130 is formed from a base substrate 132 and includes an insulating layer 134 disposed on at least one side of base substrate 132, and a conductive heater trace 136 disposed on insulating layer 134 on at least one side of base substrate 132. Conductive heater trace 136 extends distally along body 131a of thermal cutting element 130 and loops back proximally such that first and second ends 138, 140 of conductive heater trace 136 are disposed at proximal extension 131b of thermal cutting element 130. First and second contact clips 139, 141 (or other suitable electrical connections) are coupled to proximal extension 131b of thermal cutting element 130 in electrical communication with first and second ends 138, 140, respectively, of conductive heater trace 136 for connecting lead wires (not shown) to thermal cutting element 130 to enable application of an AC voltage thereto to heat thermal cutting element 130, e.g., via resistive heating. More specifically, the lead wires electrically connect thermal cutting element 130 to second activation switch 90 (FIG. 1) and electrosurgical generator “G” (FIG. 1) to enable selective activation of the supply of an AC voltage to thermal cutting element 130 for heating thermal cutting element 130 to heat and thereby thermally cut tissue. Thermal cutting element 130 may be configured to cut previously (or concurrently) sealed tissue grasped between jaw members 110, 120, tissue extending across jaw member 120, tissue adjacent the distal end of jaw member 120, etc. In addition to or as an alternative to cutting, thermal cutting element 130 may be configured for other tissue treatment, e.g., coagulation.

Base substrate 132 may be formed from any suitable material such as, for example, stainless steel, aluminum, aluminum alloys, titanium, titanium alloys, other suitable materials, combinations thereof, etc. Base substrate 132 may be formed via laser cutting, machining, casting, forging, fine-blanking, or any other suitable method. Base substrate 132 may define a thickness of, in aspects, from about 0.003 in to about 0.030 in; in other aspects, from about 0.004 in to about 0.015 in; and in still other aspects, from about 0.005 in to about 0.012 in.

Insulating layer 134, as noted above, may be disposed on either or both sides of base substrate 132. Insulating layer 134 may be a Plasma Electrolytic Oxidation (PEO) coating formed via PEO of either or both sides of base substrate 132. Other suitable materials for insulating layer 134, e.g., PTFE, PEEK, PEI, glass, etc., and/or methods of forming insulating layer 134, e.g., anodization, deposition, spraying, adhesion, mechanical attachment, etc., on either or both sides of base substrate 132 are also contemplated. Where insulating layer 134 is disposed on both sides of base substrate 132, the sides may be of the same or different materials and/or of the same or different thicknesses. Insulating layer 134 may define a thickness (on either or both sides of base substrate 132), in aspects, from about 0.0005 in to about 0.0015 in; in other aspects, from about 0.0007 in to about 0.0013 in; and in still other aspects, from about 0.0009 in to about 0.0012 in. In aspects wherein an insulating base substrate 132, e.g., ceramic, is utilized, insulating layer 134 may be omitted. Further, in aspects, multiple insulating layers 134 are provided on the same side, e.g., two insulating layers 134 on top of one another, each of which may define a thickness (similar or different from one another) within the above-noted ranges or which may collectively define a thickness within the above-noted ranges.

Conductive heater trace 136, as noted above, is disposed on insulating layer 134 (or directly on base substrate 132 where base substrate 132 itself is insulating) on one side of thermal cutting element 130, although it is also contemplated that conductive heater trace 136 extend to the other side of thermal cutting element 130 or that a second conductive heater trace 136 be provided on the other side of thermal cutting element 130. Conductive heater trace 136 may be formed from, for example, platinum, nichrome, kanthal, combinations thereof, or other suitable metal(s) and is disposed on insulating layer 134 via a deposition process, e.g., sputtering, via screen printing, via sintering, or in any other suitable manner. Conductive heater trace 136 may define a thickness, in aspects, from about 0.0002 in to about 0.0030 in; in other aspects, from about 0.0006 in to about 0.002 in; and in still other aspects, from about 0.0008 in to about 0.0012 in.

In aspects, thermal cutting element 130 further includes an encapsulating layer 138 disposed on either or both sides of body 131a of thermal cutting element 130 and/or proximal extension 131b of thermal cutting element 130. For example, encapsulating layer 138 may encapsulate body 131a of thermal cutting element 130 on the side of thermal cutting element 130 including an insulating layer 134 and conductive heater trace 136, although other configurations are also contemplated. Encapsulating layer 138 may define a thickness (on either or both sides of base substrate 132), in aspects, from about 0.0005 in to about 0.0015 in; in other aspects, from about 0.0007 in to about 0.0013 in; and in still other aspects, from about 0.0009 in to about 0.0012 in.

Thermal cutting element 130 as a whole (e.g., including base substrate 132, one or more insulating layers 134 on either or both sides, conductive heater trace 136, and encapsulating layer 138 on either or both sides) may define a thickness, in aspects, from about 0.010 in to about 0.018 in; in other aspects, from about 0.011 to about 0.016 in; and in still other aspects, from about 0.013 in to about 0.015 in.

In configurations where thermal cutting element 130 is double-sided, e.g., includes, on each side, one more insulating layers 134, a conductive heater trace 136, and an encapsulating layer 138, the conductive heater traces 136 on the first and second sides can be connected through, around, or via the thermal cutting element 130. For example, the insulative layer 134 on the first side may have an opening towards a distal end thereof to expose the base substrate 132, enabling the first conductive heater trace 136 to make connection thereto. Correspondingly, the insulative layer 134 on the second side may also have an opening towards a distal end thereof to expose the base substrate 132, enabling the second conductive heater trace 136 to make connection thereto. In such aspects, the base substrate 132 is made at least partially from an electrically conductive material and thus becomes an electrically conductive pathway, e.g., a via, between the first and second conductive heater traces 136. This configuration provides a thermal heater trace loop that starts towards the proximal end of the first side of the thermal cutting element 130, extends distally along the first side, connects through towards the distal end of the second side, and extends proximally along the second side towards the proximal end thereof. Thus, the contacts for connection to the first and second contact clips 139, 141 are provided on opposite sides of the thermal cutting element 130.

Referring still to FIGS. 4 and 5, thermal cutting element 130 may be configured to receive an applied voltage (V_{AC}), e.g., the voltage output from electrosurgical generator “G” (FIG. 1) to thermal cutting element 130, in aspects, from about 5 volts to about 250 volts; in other aspects, from about 10 volts to about 175 volts; and in still other aspects, from about 25 volts to about 100 volts.

Thermal cutting element 130 may be configured to operate in one or more different modes, e.g., controllable/settable at electrosurgical generator “G” (FIG. 1) or on housing 20 (FIG. 1) such as, for example, adjacent to or incorporated with second activation switch 90 (FIG. 1). More specifically, thermal cutting element 130 may have a single operating mode and corresponding operating temperature for all functions, or may have multiple operating modes each having a corresponding operating temperature for one or more functions such as, for example: back scoring, tenting, plunger cutting, jaws open cutting, jaws closed cutting, slow cutting, fast cutting, spot coagulation, etc. The operating temperatures for the one or more operating modes may be similar or different and any or all may be, in aspects, of at least about 350° C.; in other aspects, from about 350° C. to about 550° C.; in yet other aspects, about or at least 550° C.; in still yet other aspects, from about 400° C. to about 500° C.; and in other aspects, from about 425° C. to about 475° C.

A difference between the resistance of thermal cutting element 130 at room temperature, e.g., 20° C., and an operating temperature, e.g., 550° C., may be, in aspects, from about 5 ohms to about 1500 ohms; in other aspects, from about 10 ohms to about 1000 ohms; and in still other aspects, from about 20 ohms to about 400 ohms.

A Temperature Coefficient of Resistance (TCR) of thermal cutting element 130 may be, in aspects, at least 50 ppm/° C.; in other aspects, at least 900 ppm/° C.; and in still other aspects, at least 3000 ppm/° C.

The power (W) output, e.g., from electrosurgical generator “G” (FIG. 1), to thermal cutting element 130 at the operating temperature of thermal cutting element 130, e.g., 550° C., may be, in aspects, at most 50 W; in other aspects, at most 40 W; and in still other aspects, at most 32 W. The initial power (W) output, e.g., from electrosurgical generator “G” (FIG. 1), to thermal cutting element 130 to reach the operating temperature may be, in aspects, at most 100 W; in other aspects, at most 75 W; and in still other aspects, at most 50 W.

Various different values and ranges for the configuration and operating parameters of thermal cutting element 130 are detailed above. The present disclosure also specifically contemplates any and all combinations of these values and/or ranges as well as any and all ratios and/or ratio ranges of the values and/or ranges of two or more of these operating parameters. For example, appropriate materials, thicknesses, and/or operating parameters may be selected such that, in aspects, thermal cutting element 130 defines a configuration that maximizes the difference between the resistance of thermal cutting element 130 at room temperature and at the operating temperature and, at the same time, minimizes the applied voltage (V_{AC}), all while enabling thermal cutting element 130 to reach a suitable operating temperature. Other optimizations are also contemplated.

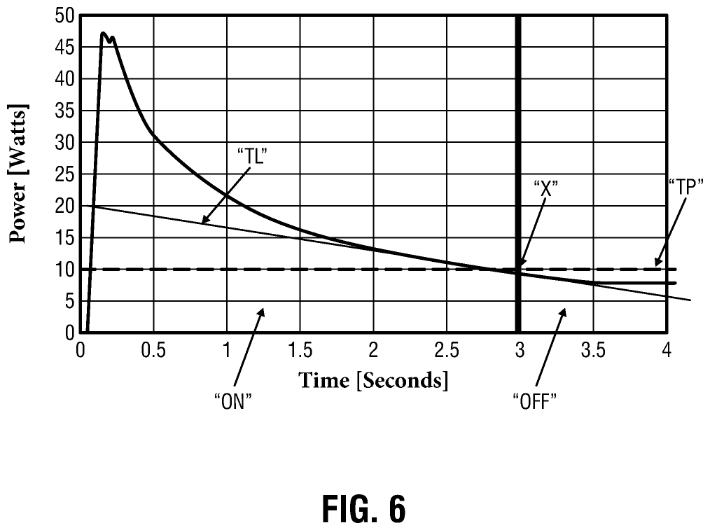

Turning to FIG. 6, in conjunction with FIGS. 1 and 4, in use, it may be desired to cut tissue such as, for example: after sealing tissue grasped between jaw members 110, 120 while maintaining the grasp on tissue; with tissue extending over jaw member 120 in a static, jaws open condition; with jaw members 110, 120 moving relative to tissue to be cut in a jaws open condition; with jaw members 110, 120 moving relative to tissue to be cut in a jaws closed condition; etc. In any or all of these or other situations where it is desired to cut tissue (or otherwise treat tissue using thermal cutting element 130), second activation switch 90 (FIG. 1) is activated to initiate the supply of energy, e.g. the AC voltage signal from electrosurgical generator “G” (FIG. 1), to thermal cutting element 130 to thereby heat thermal cutting element 130. Initially, power ramps up rapidly (approaching instantaneously but within the limits of system components and the laws of physics) to heat thermal cutting element 130 to the operating temperature in minimal time which, in turn, begins to heat tissue in contact or adjacent to thermal cutting element 130. After this initial power increase to reach the operating temperature, the power decreases. More specifically, the power required to maintain thermal cutting element 130 at the operating temperature decreases as thermal cutting element 130 continues to heat tissue and begins to cut through the heated tissue.

Cut completion, e.g., when tissue has been fully divided, may be determined by monitoring this decrease in power, e.g., after the initial rapid increase in power output. For example, cut completion can be determined when the power decreases to a threshold power “TP,” e.g., at point “X,” such that, after this threshold power “TP” is reached, the supply of energy to thermal cutting element 130 is turned off. Thus, the initial ramp and subsequent decrease in power prior to reaching the threshold power “TP” corresponds to a tissue cutting or “ON” condition and, once the threshold power “TP” is reached at point “X,” energy supply is stopped corresponding to an “OFF” condition. It is noted that the power curve illustrated in FIG. 6 continues after the threshold power “TP” is reached (and, thus, into the “OFF” condition) for illustrative purposes; however, in use, this would not be the case as the power would drop rapidly to zero once the supply of energy is stopped upon reaching the threshold power “TP.”

As an alternative to or in addition to monitoring power output relative to a threshold power “TP” to determine cut completion and, thus, to determine when to stop the supply of energy to thermal cutting element 130, cut completion can be determined by comparing power over time, e.g., a slope of the power curve, to a sloped threshold line “TL” such that, when the power curve intersects the threshold line “TL,” e.g., at point “X,” the supply of energy to thermal cutting element 130 is turned off. This intersection point “X” between the power curve and the threshold line “TL” corresponds to the point at which the slope of the power curve equals the slope of the threshold line “TL” (thus, the threshold line “TL” may be selected at least in accordance with a target slope); prior to intersection, the slope of the power curve is steeper than that of the threshold line “TL” and after intersection, the slope of the power curve is flatter than that of the threshold line “TL.”

In aspects, rather than (or in addition to) turning off the supply of energy to thermal cutting element 130 upon reaching the threshold power “TP” and/or intersecting the threshold line “TL,” an indicator, e.g., an audible tone, visual icon, tactile feedback, etc., may be provided to the operator to indicate that cut completion has been determined.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A thermal cutting element of an electrosurgical instrument, comprising:
a base substrate defining a first side and a second side;
at least one insulating layer disposed on at least the first side of the base substrate; and
a heater circuit trace disposed on the at least one insulating layer,
wherein the thermal cutting element has an operating temperature of at least about 350° C. and a Temperature Coefficient of Resistance (TCR) of at least about 50 ppm/° C.

2. The thermal cutting element according to claim 1, wherein the thermal cutting element has a TCR of at least about 900 ppm/° C.

3. The thermal cutting element according to claim 1, wherein the thermal cutting element has a TCR of at least about 3000 ppm/° C.

4. The thermal cutting element according to claim 1, wherein the operating temperature is at least about 400° C.

5. The thermal cutting element according to claim 1, wherein the operating temperature is at least about 450° C.

6. The thermal cutting element according to claim 1, wherein a difference between a resistance of the thermal cutting element at room temperature and at the operating temperature is from about 10 ohms to about 1000 ohms.

7. The thermal cutting element according to claim 1, wherein a difference between a resistance of the thermal cutting element at room temperature and at the operating temperature is from about 20 ohms to about 400 ohms.

8. The thermal cutting element according to claim 1, wherein an applied voltage of from about 10 volts to about 175 volts is required to at least one of heat the thermal cutting element to the operating temperature or maintain the thermal cutting element at the operating temperature.

9. The thermal cutting element according to claim 1, wherein an applied voltage of from about 25 volts to about 100 volts is required to at least one of heat the thermal cutting element to the operating temperature or maintain the thermal cutting element at the operating temperature.

10. The thermal cutting element according to claim 1, further comprising an encapsulating layer disposed on the first side of the base substrate.

11. An electrosurgical instrument, comprising:
first and second jaw members each defining a tissue treating surface, the first and second jaw members pivotably coupled to one another such that at least one of the first or second jaw members is movable relative to the other from a spaced-apart position to an approximated position to grasp tissue between the tissue treating surfaces, the tissue treating surfaces adapted to connect to a source of energy to treat tissue grasped therebetween, one of the first or second jaw members including a thermal cutting element extending from the tissue treating surface thereof, the thermal cutting element including:
a base substrate defining a first side and a second side;
at least one insulating layer disposed on at least the first side of the base substrate; and
a heater circuit trace disposed on the at least one insulating layer,
wherein the thermal cutting element has an operating temperature of at least about 350° C. and a Temperature Coefficient of Resistance (TCR) of at least about 50 ppm/° C.

12. The electrosurgical instrument according to claim 1, wherein the thermal cutting element has a TCR of at least about 900 ppm/° C.

13. The electrosurgical instrument according to claim 1, wherein the operating temperature is at least about 400° C.

14. The electrosurgical instrument according to claim 1, wherein a difference between a resistance of the thermal cutting element at room temperature and at the operating temperature is from about 10 ohms to about 1000 ohms.

15. The electrosurgical instrument according to claim 1, wherein an applied voltage of from about 10 volts to about 175 volts is required to at least one of heat the thermal cutting element to the operating temperature or maintain the thermal cutting element at the operating temperature.

16. An electrosurgical system, comprising:
an electrosurgical generator; and
a thermal cutting element, the thermal cutting element including:
a base substrate defining a first side and a second side;
at least one insulating layer disposed on at least the first side of the base substrate; and
a heater circuit trace disposed on the at least one insulating layer,
wherein the thermal cutting element has an operating temperature of at least about 350° C. and a Temperature Coefficient of Resistance (TCR) of at least about 50 ppm/° C., and
wherein the electrosurgical generator is configured to supply an AC voltage signal to the heater circuit trace to at least one of heat the thermal cutting element to the operating temperature or maintain the thermal cutting element at the operating temperature.

17. The electrosurgical system according to claim 16, wherein the electrosurgical generator is configured to monitor power during the supply of the AC voltage signal and to at least one of provide an indicator or stop the AC voltage signal when the power declines to a threshold power level.

18. The electrosurgical system according to claim 16, wherein the electrosurgical generator is configured to monitor power slope during the supply of the AC voltage signal and to at least one of provide an indicator or stop the AC voltage signal when the power slope flattens to a power slope threshold.

19. The electrosurgical system according to claim 16, wherein the AC voltage signal has an applied voltage of from about 10 volts to about 175 volts.

20. The electrosurgical system according to claim 16, wherein the electrosurgical generator is configured to provide a maximum power associated with the AC voltage signal of about 50 W.
